SEQUENCE LISTING (12 sequences)

SEQ ID NO: 1 - DNA, 29727 bp, SARS coronavirus Urbani
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac

SEQ ID NO: 2 - DNA, 353 bp, SARS coronavirus Urbani
  misc_feature (1)..(353): Portion of SARS CoV genome amplified by IMCB-1 primer set
acatcaaattgcgctaagagattagcacaacgtgtgtttaacaattatatgccttatgtgtttacattattgttccaattgtgttacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattacaattctctaacgccattac

SEQ ID NO: 3 - DNA, 19 bp, Artificial Sequence
  PCR primer IMCB-1U
acatcaaattgcgctaaga

SEQ ID NO: 4 - DNA, 21 bp, Artificial Sequence
  PCR primer IMCB-1L
acaattctctaacgccattac

SEQ ID NO: 5 - DNA, 156 bp, SARS coronavirus Urbani
  misc_feature (1)..(156): Portion of SARS CoV genome amplified by IMCB-2 primer set
gccgtagtgtcagtatcatcaccagatgctgttactacatataatggatacctcacttgtcatcaaagacatctgaggagcactttgtagaaacagtttctttcgctggctcttacagagattggtcctattcagcacctaactctgtacgctgtc

SEQ ID NO: 6 - DNA, 19 bp, Artificial Sequence
  PCR primer IMCB-2U
gccgtagtgtcagtatcat

SEQ ID NO: 7 - DNA, 21 bp, Artificial Sequence
  PCR primer IMCB-2L
cacctaactctgtacgctgtc

SEQ ID NO: 8 - DNA, 78 bp, SARS coronavirus Urbani
  misc_feature (1)..(78): Portion of SARS CoV genome amplified by IMCB primer set 3
gcactttgtagaaacagtttctttggctggctcttacagagattggtcctattccacctaactctgtacgctgtcctg

SEQ ID NO: 9 - DNA, 26 bp, Artificial Sequence
  PCR primer IMCB-3U
gcactttgtagaaacagtttctttgg

SEQ ID NO: 10 - DNA, 24 bp, Artificial Sequence
  PCR primer IMCB-3L
cacctaactctgtacgctgtcctg

SEQ ID NO: 11 - DNA, 17 bp, Artificial Sequence
  Probe for IMCB-3 primer amplification product
tggctcttacagagatt

SEQ ID NO: 12 - DNA, 157 bp, SARS coronavirus Urbani
  misc_feature (1)..(157): portion of the SARS CoV genome amplified by the IMCB-3 primer set (bottom strand in Figure 2)
cacctaactctgtacgctgtcctgaataggaccaatctctgtaagagccagccaaagaaactgtttctacaaagtgctcctcagatgtctttgatgacgaagtgaggtatccattatatgtagtaacagcatctggtgatgatactgacactacggc

(19) United States
(12) Patent Application Publication
(10) Pub. No.: US 2007/0248949 A1
(43) Pub. Date: Oct. 25, 2007

(54) Sensitive and Specific Test to Detect Sars Coronavirus

(75) Inventors: Masafumi Inoue, Singapore (SG); Wanjin Hong, Singapore (SG)

Correspondence Address: BIRCH STEWART KOLASCH & BIRCH, PO BOX 747, FALLS CHURCH, VA, 22040-0747, US

(73) Assignee: AGENCY FOR SCIENCE, TECHNOLOGY AND RESEARCH, CENTROS (SG)

(21) Appl. No.: 10/582,969

(22) PCT Filed: Dec. 17, 2004
(86) PCT No.: PCT/SG04/00416

§ 371 Date: Sep. 19, 2006

Related U.S. Application Data
(60) Provisional application No. 60/529,737, filed on Dec. 17, 2003.

(51) Int. Cl. C12Q 1/68; C12Q 1/70

(52) U.S. Cl. 435/5; 435/6

(57) ABSTRACT

The invention provides a simple, specific and sensitive test for the presence of SARS coronavirus. Kits comprising primers useful in the test are also provided.

### RELATED APPLICATIONS

The present application claims priority of U.S. Provisional Application No. 60/529,737 filed Dec. 17, 2003.

### SEQUENCE LISTING

The present application includes an appended Sequence Listing of 20 pages presenting 12 sequences.

### FIELD OF THE INVENTION

The instant invention provides a qualitative nucleic acid amplification assay for the detection of SARS coronavirus in patient samples. The assay uses primer pairs that have been developed that provide excellent sensitivity and specificity for detection of SARS coronavirus.

### BACKGROUND OF THE INVENTION

An outbreak of atypical pneumonia, severe acute respiratory syndrome (SARS) is thought to have originated from Guang-dong Province, Republic of China in late 2002. The mortality rate of individuals suffering from SARS can be as high as 15%, depending on the age group analyzed. SARS is a highly infectious and acute condition with an extremely high mortality rate. The condition is caused by a human coronavirus, named SARS coronavirus (SARS coronavirus). The disease killed 774 patients out of 8098 probable SARS cases from November 2002 to July 2003, and has had a profound economic and social impact globally.

In many viral diseases, the spread of the virus is greatest during the early symptomatic phase that is around and immediately following the onset of symptoms. Unfortunately, virus excretion is comparatively low during the initial phase of SARS. It peaks in respiratory specimens and in stools at around day 10 after the onset of the clinical illness. In order to make an early diagnosis, it is therefore necessary to use highly sensitive tests that are able to detect the low levels of viral genome present during the first days of the illness.

There are many non-standardized and sensitive tests under development in many countries. The available SARS RT-PCR based diagnostic tests often suffer the drawback of being complex and difficult to administer. The typical SARS diagnostic test uses nested (two step) polymerase chain reaction (PCR) to accomplish a certain level of specificity and sensitivity. See, e.g., “SARS-COV Specific RT-PCR Primers”, by William J. Bellini, Ph.D. Chief, Measles Virus, Section DVRD/NC1D/CDC, CDCprimers.pdf, obtainable from th World Health Organization (WHO), which is hereby incorporated by reference in its entirety, for description of the typical PCR test for SARS.

### BRIEF DESCRIPTION OF THE DRAWINGS

FIG. 1 shows the portions of the SARS coronavirus genome amplified by the IMCB primer sets.

FIG. 2 shows the portion of the SARS coronavirus genome amplified by the IMCB-3 primer set and aligns the IMCB-3 primers and the IMCB-3 probe along the sequence of the SARS coronavirus genome. The upper strand sequence is shown as nucleotides 4609-4765 of SEQ ID NO: 1. The lower strand is shown as SEQ ID NO: 12.

FIGS. 3A-3C show gels demonstrating the efficacy of the primers of the invention. FIG. 3A shows the detection of SARS coronavirus using the IMCB-2 primer set where the virus copy number per sample loaded varies from 26.1 copies to 0.07 copies. Lane 1 is a marker, lanes 2 & 3 contain 26.1 copies of the virus per 5 μl, lanes 4 & 5 contain 12.6 copies of the virus per 5 μl, lanes 6 & 7 contain 1.96 copies of the virus per 5 μl, lanes 8 & 9 contain 2.0 copies of the virus per 5 μl, lanes 10 & 11 contain 0.07 copies of the virus per 5 μl, lane 12 contains a negative control of an unrelated virus and lane 13 contains another marker.

FIG. 3B shows a second experiment detecting SARS coronavirus using the IMCB-2 primer set, where the virus copy number per sample loaded varies from 26.1 copies to 0.08 copies. Lane 1 is a marker, lanes 2 & 3 contain 26.1 copies of the virus per 5 μl, lanes 4 & 5 contain 8.2 copies of the virus per 5 μl, lanes 6 & 7 contain 2. 6 copies of the virus per 5 μl, lane 8 contains 0.8 copies of the virus per 5 μl, lane 9 contains 0.25 copies of the virus per 5 μl, lanes 10 & 11 contain 0.08 copies of the virus per 5 μl, lane 12 contains a negative control of an unrelated virus and lane 13 contains another marker.

FIG. 3C shows the amplified product from a sample containing 5 copies of SARS coronavirus genomic RNA per run in a total of 5 μl (duplicated). The product is resolved by 3% agarose gel electrophoresis. A 10% of total reaction volume (5 μl) is loaded per lane. Lane 1, amplified product; lane 2, amplified product of a duplicate reaction; lane M, 100 bp ladder.

FIGS. 4A to 4C show the sensitivity achieved using the present invention to detect SARS coronavirus nucleic acid with the primer set IMCB-1. FIG. 4A shows results achieved with 8.8 pfu (2200 copies) per sample (lanes 1 and 2) to 0.08 pfu (22 copies) per sample (lanes 5-6). Lanes 7 and 8 show a no virus control. FIG. 4B shows the results of another run of the same assay using from 0.08 pfu (22 copies) per sample to 0.0008 pfu (0.2 copies) per sample. Lanes 11 and 12 show a no virus control. FIG. 4C shows a third run using from 0.08 pfu (22 copies) per sample to 0.004 pfu (1 copy) per sample. Lanes 7 and 8 are a no virus control sample. M is a molecular length marker.

### DESCRIPTION OF THE INVENTION

At the time of the original SARS outbreak there was a lack of rapid detection. Sensitive and specific rapid detection would have allowed quick diagnosis of infected patients to enable better containment of the spread of the epidemic. A PCR-based assay was developed at the Bernhard Nocht Institute (BNI) (Drosten et al, 2003) and is available commercially from Artus. The primers identified by BNI and used by Artus are from the SARS coronavirus non-structural protein 9 that encodes an RNA polymerase. According to WHO recommendations, results of these tests should still not be used to rule out a suspected case of SARS (WHO Update 71).

Because presently available tests are not generally able to detect the requisite small amounts of SARS coronavirus (SARS coronavirus), they do not yet play a role in patient management and case control, as SARS patients may be capable of infecting others during the initial phase and therefore need to be reliably detected and quickly isolated.

Coronoviruses are a family of RNA viruses with a large envelope that propagate in the cytoplasm of host cells and usually cause mild respiratory disease in man and animals.

The SARS Coronavirus has been isolated and sequenced. A prototype sequence of 29,727 basepairs can be found at GENBANK, under Accession No. AY278741, hereby incorporated by reference and presented also as SEQ ID NO: 1. See also, Y. J. Ruan et al., Lancet 361:1779-1785 (2003), analyzing the genome sequence of 14 different isolates, and P. A. and Rota et al., Science 300:1377-1378 (2003), characterizing one of the first isolates to be associated with SARS.

Sequencing of the complete genome of the SARS virus from a number of different isolates has indicated that the virus has a typical coronavirus genome organization, but that the virus is not closely related to any other known coronaviruses.

The SARS virus encodes 14 open reading frames (ORFs), including the replicase 1a and 1b proteins and four structural proteins, spike protein (S), envelope protein (E), membrane protein (M) and nucleocapsid protein (N).

Ruan et al. (2003, supra) compared the genome sequence of a Singapore case of SARS coronavirus and a database of other coronavirus genomic sequences. From this they were able to find which regions of SARS coronavirus were homologous to other coronavirus and thus conserved among coronavirus strains and which sequences were unique to SARS coronavirus Singapore strain SIN2500.

The instant invention provides a simple, sensitive and specific diagnostic test. This test provides a simple yet sensitive and specific nucleic acid amplification system compared with others that have so far been developed. By use of the primers described herein, the instant invention is made more sensitive and specific than the detection methods of the prior art. Furthermore, such specificity and sensitivity may be enhanced by using a one step PCR method, instead of a two step PCR.

The present invention utilizes specific primer pairs designed from the SARS coronavirus non-structural protein 1 (NSP1) a putative proteinase. These primers can be used with many techniques to detect the presence of SARS coronavirus.

In one embodiment the instant invention provides a simple gel-based RT-PCR detection kit. Such a kit will include one or more primers and/or probes according to the invention, for example a kit may contain primers consisting of one or more polynucleotides comprising a nucleotide sequence of SEQ ID NOs: 3, 4, 6, 7, 9, 10 and 11. A kit according to the invention may optionally include a positive control nucleic acid, for example a SARS coronavirus genomic nucleic acid, or at least a portion thereof comprising the NSP1 region, as either RNA or DNA.

The present invention also provides a method for detecting SARS coronavirus nucleic acid in a sample. The method may be generally described as comprising amplifying a nucleic acid of a sample with a reverse transcriptase and at least one primer specific for the NSP1 region of a SARS coronavirus to generate a nucleic acid ampification product. The amplification product is then analyzed, seeking to detect an expected nucleic acid amplification product. Detection of the expected product indicates the presence of SARS coronavirus nucleic acid in the sample.

The primers of the instant invention can also be used as a primer set for real-time PCR detection using PCR platforms such as the Roche LightCycler™, the Stratagene Real-time PCR system, the Applied Biosystems ABI 7000 real time PCR analyzer or any other suitable detection platform.

Specific primer pairs have been designed by comparing conserved regions among SARS coronavirus strains and avoiding sequence regions that were conserved among all coronaviruses generally. A portion of the SARS coronavirus genome that encodes the NSP1 proteinase was selected by this comparison as a detection target.

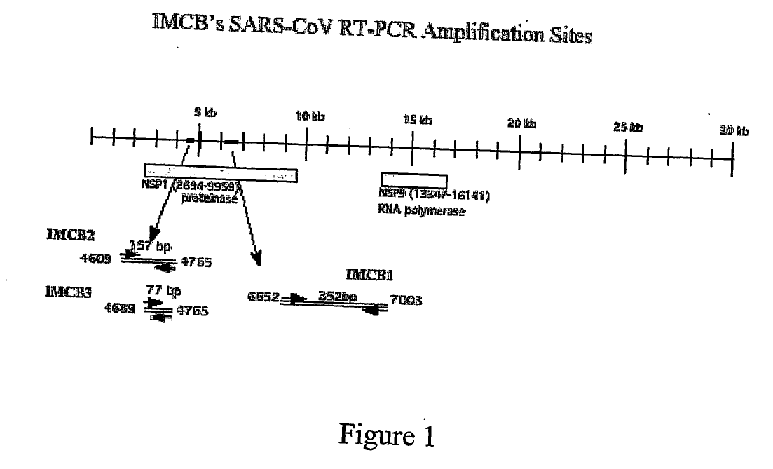

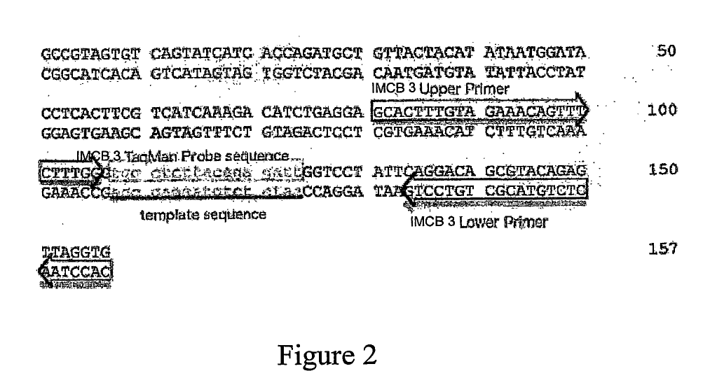

As illustrated in FIG. 1, the primers of the present invention hybridize in the portion of the SARS coronavirus genome from about nucleotide number 2200 to about nucleotide 9800 in a manner as to amplify this region, or a portion thereof. This region is known as the NSP1 region, which codes for a putative proteinase. This region was chosen as a putative region to amplify because unlike the RNA polymerase region utilized in prior art assays, there is a significant portion of it in which no mutations appear to have occurred among SARS coronaviruses and this region is very specific to all isolates of SARS coronavirus; a hypothesis which has been examined by an NCBI (National Center for Biotechnology Information) Blast search. In the region from about nucleotide 2650 to about nucleotide 7850 has been identified that appears to bear no mutations among several strains (Ruan et al., 2003). A preferred part of the NSP1 region for amplification is the part from and including nucleotide 4609 to and including nucleotide 7003.

The primers of the invention should be at least 16 nucleotides in length, more preferably at least 18 nucleotides in length, still more preferably at least 20 nucleotides in length. The primers should be less than 50, preferably less than 30, more preferably less than 25 nucleotides in length, so as to preserve the specificity of the primers.

Thus, the SARS detection method of the invention lies generally in use of a set of primers that are specific for the NSP1 region of the SARS coronavirus genome for PCR amplification of this part of the genome and detection of the amplification product. The method of the invention can be performed, for example by amplifying nucleic acids present in the sample using a forward primer and a reverse primer selective for the region of the SARS genome from nucleotide 6652 to nucleotide 7003, or using a forward primer and a reverse primer selective for the region of the SARS genome from nucleotide 4609 to nucleotide 4765, said primers having a certain primer length in nucleotides and being separated by a separation length that is a certain number of nucleotides, to obtain an amplification product. The amplification product is then detected. The amplification product can be detected, for example, by determining the length of the amplification product in nucleotides, either by a chromatographic method or by a gel electrophoretic method, e.g by electrophoresis in 2 or 3% agarose. The presence of an amplification product having a length in nucleotides that is the sum of the forward primer length, the reverse primer length and the separation length indicates the presence of SARS coronavirus nucleic acid in the sample.

Alternatively, the product can be detected using a hybridization probe, for example using real-time fluorescent detection in the Taqman™ system. The hybridization probe preferably comprises a nucleotide sequence that is the same as that of a portion of the amplification product that would be obtained using the amplification primers selected and a SARS coronavirus genomic nucleic acid as a template. The hybridization probe should be at least 16 nucleotides in length, more preferably at least 18 nucleotides in length, still more preferably at least 20 nucleotides in length. The probe should be less than 50, preferably less than 30, more preferably less than 25 nucleotides in length, so as to preserve the specificity of the probe.

The essential function of a primer or probe according to the present invention is to specifically hybridize to a SARS coronavirus nucleic acid, either an RNA or DNA, and not to cross-hybridize to other cororiavirus nucleic acids or to nucleic acids of other viruses. Thus, a primer or probe according to the present invention “consists essentially of” a nucleotide sequence if it includes that sequence and additional nucleotides that do not impair the ability of the primer or probe to specifically hybridize to a SARS coronavirus nucleic acid under the conditions selected for performing a diagnostic assay according to the invention.

Materials and Methods Generally Used in the Examples

SARS Coronavirus Culture

The SARS coronavirus isolate (2003VA2774), which has been previously sequenced (Ruan et al, 2003), is used for this study. The virus stock is propagated in Vero E6 cells (ATCC: C1008) with medium 199 (Sigma Aldrich, USA) supplemented with 5% fetal calf serum (FCS) (Biological Industries, Israel). When more than 75% of the cell monolayer showed cytopathic effects, the culture supernatant is harvested, clarified by centrifugation at 1300×g, aliquoted and stored at −80° C. until use. The PFU of the current preparation is determined as 1×10(7) PFU/ml.

Plaque Assay

A plaque assay is carried out to determine the virus titer in the culture. Briefly, 100 ml series of 10-fold dilution of the virus stock is added to a confluent monolayer of Vero E6 cells in a 24-well plate and incubated for 1 hour at 37° C.

Following this, 1 ml of 1% carboxymethylcellulose overlay in medium 199 with 5% FCS is then added to each well. After 4 days of incubation at 37° C. in 5% CO₂, the cells are then fixed with 10% formalin and stained with 2% crystal violet. The plaques are counted visually and the virus titer determined.

RNA Extraction

SARS Coronavirus Standards:

10-fold dilution of the stock virus is prepared in serum obtained from a healthy volunteer. RNA is extracted using the QIAGEN Viral RNA Kit (QIAGEN GMbH, Germany) according to the instructions given in the product insert.

Patient Specimens:

Virus isolation is performed on a bronchoalveolar lavage specimen of SARS cases belonging to the original case cluster from Singapore. RNA is directly extracted from the specimen using a Qiagen QIAamp viral RNA extraction kit (catalog no. 52906) according to the instructions given in the product insert.

Other Viruses:

RNA is directly extracted from the stock vial obtained from ATCC (VA, USA) using the QIAGEN Viral RNA Mini Kit (QIAGEN GMbH, Germany) according to the instructions given in the product insert.

MRC-5 Cell Line:

Total RNA is extracted directly from the normal diploid human fibroblast cell line MPC-5 (ATCC CCL171) using a Qiagen RNA extraction kit (catalog no. 74104) and RNA is quantitated using a spectrophotometer.

### EXAMPLE 1

### Primer Design

Primers are selected using the sequence of the SARS coronavirus Urbani strain (AY278741) based on conserved regions of SARS coronavirus genome (Rota et al. 2003). A set of primer pairs in the proteinase gene region (position 6652-7003, of a non-structural protein 1 (NSP1) region) is found to be most suitable as it exhibits the lowest cross homology with other viruses (Ruan et al. 2003). The primers are designed to take into account possible mismatches throughout the genome and to avoid or at least minimize primer dimer formation.

The NSP1 region (proteinase) target of the primers is generally well conserved among isolates of SARS coronavirus. In most of the NSP1 region, there is no match between a SARS fragment and other coronaviruses compared with NSP9 (RNA polymerase), which has very strong homology across the coronavirus group (Ruan et al. 2003).

In addition, no mutation has been found occurring in a significant portion of this region (Ruan et al. 2003). It is also very specific to SARS coronavirus (all known isolates), as confirmed by testing amplification of RNA samples from other viruses using primers targeting this region (Table 1) and by NSTBI BLAST search of all known sequences in the NSTBI database excluding SARS coronavirus sequences.

Three sets of primer pairs are identified from the NSP1 proteinase region. The location of these three sets in relation to the SARS coronavirus genome can be seen in FIG. 1. The first set amplifies a segment IMCB-1 that is 352 base pairs in length, SEQ ID NO: 2. This sequence is flanked by an upper primer (IMCB-1-U, SEQ ID NO: 3) and a lower primer (IMCB-1-L, SEQ ID NO: 4). These primers can be used to specifically detect the presence of SARS coronavirus nucleic acids in a sample.

| | |
|---|---|
| IMCB-1-U (19-mer): | |
| 5′ ACATCAAATTGCGCTAAGA3′ | (SEQ ID NO: 3) |
| IMCB-1-L (21-mer): | |
| 5′ ACAATTCTCTAACGCCATTAC3′ | (SEQ ID NO: 4) |

Set 2 relates to a fragment called IMCB-2 of 157 base pairs in length, SEQ ID NO: 5. This sequence is flanked by an upper primer (IMCB-2-U, SEQ ID NO: 6) and a lower primer (IMCB-2-L, SEQ ID NO: 7). These primers can be used in like manner to the IMCB-1 primer set to specifically detect the presence of SARS coronavirus nucleic acids in a sample. Both the IMCB-1 and IMCB-2 primer sets can be used with the reagents and conditions set forth, for instance, in the One-Step RT-PCR described in Example 3.

| | |
|---|---|
| IMCB-2-U (19-mer): | |
| 5′ GCCGTAGTGTCAGTATCAT3′ | (SEQ ID NO: 6) |
| IMCB-2-L (21-mer): | |
| 5′ CACCTAACTCTGTACGCTGTC3′ | (SEQ ID NO: 7) |

Set 3 relates to a fragment called IMCB-3 that is a portion of IMCB-2 that is 77 base pairs in length, SEQ ID NO: 8. This sequence is flanked by an upper primer (IMCB-3-U, SEQ ID NO: 9) and a lower primer (IMCB-3-L, SEQ ID NO: 10). A probe oligonucleotide (IMCB-3-probe, SEQ ID NO: 11) is situated between the two primers of IMCB-3. The primers and the probe are used to detect the presence of SARS coronavirus, for example in a real-time assay using fluorescent detection of the amplified fragment. The lower primer of set 2, IMCB-2-L and of set 3, IMCB-3-L, are almost identical, but IMCB-3-L is 3 nucleotides longer at the 3′ end. These primers and/or probe can be used to specifically detect the presence of SARS coronavirus nucleic acids in a sample.

| | |
|---|---|
| IMCB-3-U (26-mer): | |
| 5′ GCACTTTGTAGAAACAGTTTCTTTGG3′ | (SEQ ID NO: 9) |
| IMCB-3-L (24-mer): | |
| 5′ CACCTAACTCTGTACGCTGTCCTG3′ | (SEQ ID NO: 10) |
| IMCB-3-Probe (17-mer): | |
| 5′ TGGCTCTTACAGAGATT3′ | (SEQ ID NO: 11) |

### EXAMPLE 2

### Detection of SARS Coronavirus Using the IMCB-1 Primer Pair

The first primer pair (IMCB-1) described above is designed for amplifying the portion of the SARS coronavirus genome from nucleotide 6652 to nucleotide 7003. The forward primer, also herein called an “upper primer” is a 19-mer that hybridizes to the SARS coronavirus genome beginning at position 6652 and has the sequence of SEQ ID NO: 3.

The reverse primer, called herein a “lower primer” is a 21-mer that hybridizes to the SARS coronavirus genome at position 6983 and has the sequence of SEQ ID NO: 4.

RNA is extracted from samples thought to contain SARS RNA by known methods. The RNA is then converted to DNA using a reverse transcriptase or any other method known in the art. A sample mixture is converted into cDNA in a typical manner using a 1st Strand cDNA Synthesis Kit for RT-PCR (sold by Roche, Basel, Switzerland, catalog no. 1 483 188).

The first strand cDNA reaction is carried out using the following reagents at the indicated concentrations:

| REAGENTS | FINAL CONC. | |
|---|---|---|
| 1. 10× Reaction buffer | 1× | |
| 2. MgCl₂, 25 mM | 5 | mM |
| 3. dNTP mix, 1O mM ea. | 1 | mM ea. |
| 4. Specific primer, 20 μM | 1 | μM |
| | (IMCB-1 Lower Primer) | |
| 5. RNase inhibitor | 50 | units |
| 6. AMV reverse transcriptase | 20 | units |
| 7. Gelatin (0.5 mg/ml) | 0.01 | mg/ml |

| | |
|---|---|
| 8. Sterile water | — |
| 9. RNA sample | — |

to produce a total reaction volume of 20.0 μl. The reaction is allowed to proceed at 25° C. for 10 minutes and then at 42° C. for 60 minutes.

The reverse transcriptase is then inactivated by incubating the reaction at 99° C. for 5 minutes and cooling to 4° C. for 5 minutes.

The cDNA is then amplified by adding 1 μl of 1 μg/μl of single stranded DNA to each sample and preparing the sample for PCR. It will be recognized by those of skill in the art that other methods of amplification known in the art can be performed to amplify the DNA.

PCR is performed using the following reagents and conditions. The reaction mixture is then prepared using the following reagents and concentrations.

| REAGENTS | FINAL CONC. | |
|---|---|---|
| 1. 2× master mix* | ×1 | |
| 2. Upper primer | 0.3 | μM |
| 3. Lower primer | 0.3 | μM |
| 9. DNA sample | — | |
| Total reaction volume | 50.0 | μl |

*Promega #M7501 (Madison, Wisconsin)

The thermal cycling is performed using a Stratagene (La Jolla, Calif.) Robocycler 96 for the respectively enumerated steps, temperatures and times.

| | | | | |
|---|---|---|---|---|
| 1. | Initial denature, | 95° C. | 5 | min |
| 2. | Denaturation, | 95° C. | 45 | sec |
| 3. | Annealing, | 49° C. | 80 | sec |
| 4. | Extension, | 72° C. | 50 | sec |
| | = 2,3,4 cycling: 35 = | | | |
| 5. | Final extension, | 72° C. | 3 | min |

The PCR product is analyzed on a 2.0% agarose gel. The detection can be alternatively done by an ABI (Foster City, Calif.) PRISM 7000 Sequence Detection System to confirm the presence of the correct amplified region, which is identified as a nucleic acid fragment of 352 nucleotides.

It will be obvious to a skill person in the art that the IMCB-1 primer sets can be used in the Two-Step RT-PCT as well as in the One-Step RT-PCR. In particular, the IMCB-1 primer sets may be used with the reagents and conditions set forth, for instance, in the One-Step RT-PCR described in Example 3.

### EXAMPLE 3

### SARS Coronavirus Detection Using IMCB-2 Primer Set

The second primer pair set described above is designed for amplifying the portion of the SARS coronavirus genome from nucleotide 4609 to nucleotide 4765. The forward primer, called herein “Upper Primer” or “IMCB-2-U” is a 19-mer that hybridizes to the SARS coronavirus genome beginning at position 4609 has the following sequence:

| | | |
|---|---|---|
| IMCB-2-U | 5′ GCCGTAGTGTCAGTATCAT3′ | (SEQ ID NO: 6) |

The reverse primer, called herein “Primer Lower” or “IMCB-2-L” is a 21-mer that hybridizes to the SARS coronavirus genome at position 4765 has the following sequence:

| | | |
|---|---|---|
| IMCB-2-L | 5′ CACCTAACTCTGTACGCTGTC3′ | (SEQ ID NO: 7) |

The method to perform the assay is as follows.

5 μl of RNA sample is diluted to a 50 μl reaction volume with 45 μl of a premixture solution containing reaction buffer, Q-solution (Qiagen, catalog no. 210210) and dNTP mix, at a final concentration of 400 μM each, and an upper primer and a lower primer at a final concentration of 0.6 μM each. RNase inhibiter at 10 units/reaction, enzyme mix, RNase free water is also added.

The thermal cycling is performed using a Stratagene Robocycler 40 (La Jolla, Calif.) with the following steps, reverse transcription at 50° C. for 30 min and initial denature at 95° C. for 15 min, followed by denaturation at 95° C. for 45 sec, annealing at 50° C. for 80 sec, extension at 72° C. for 50 sec. The cycle is repeated 42 times. The entire RT PCR is completed after a final extension at 72° C. for 10 min. The PCR product is analyzed by gel electrophoresis in 2.0% agarose.

With patients' samples and pre-determined SARS coronavirus standards, it is found that the detection limit of the diagnostic test of the instant invention is approximately 200 copies/ml (1 copy/5 μl reaction) for the virus as confirmed when measured using ARTUS RealArt™ HPA-Coronavirus LC RT PCR Reagents (cat No: 5601-03).

### EXAMPLE 4

### A RT-PCR SARS Diagnostic Kit and Its Use

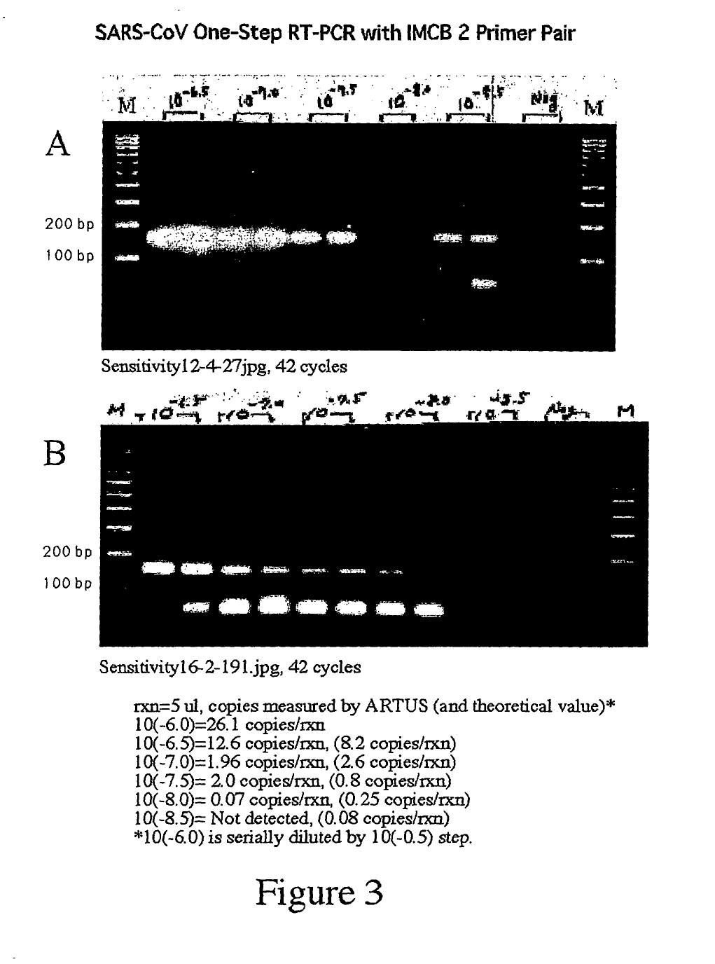

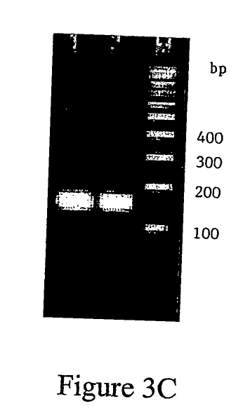

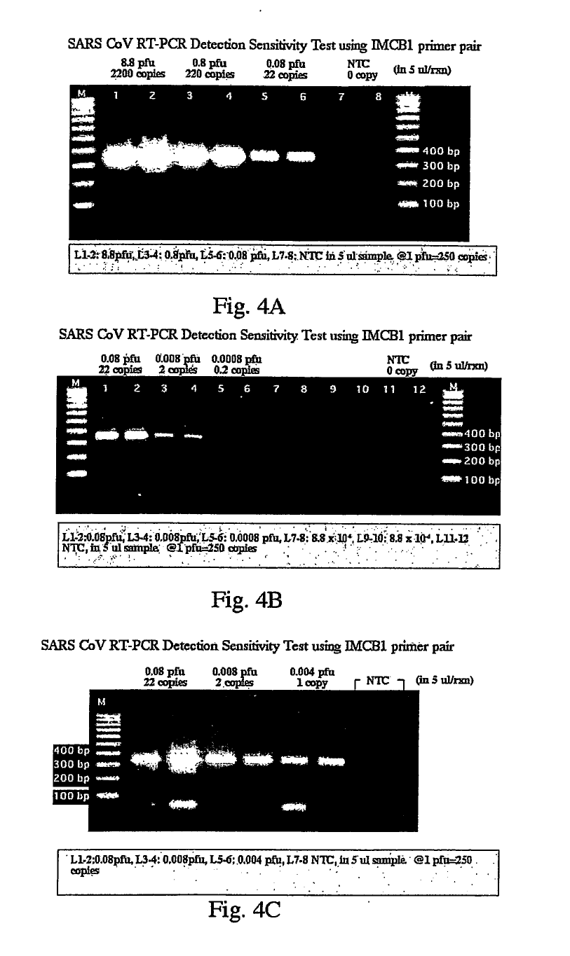

A kit according to this Example is typically prepared to contain 50 or 100 reactions. The kit is composed of the items listed below; it should be stored at −20° C. in a non-frost-free freezer. FIG. 3C shows results that are obtained using this kit with the IMCB-2 primer set.

The Reverse Transcriptase-Polymerase Chain Reaction (RT-PCR) kit described herein is used for detecting the presence of severe acute respiratory syndrome coronavirus (SARS coronavirus) RNA in samples extracted from specimens with an appropriate RNA extraction method of choice. The region of the SARS coronavirus genome amplified in this assay lies in the the NSP-1 region (proteinase) of the virus genome. This kit is optimized to detect a few molecules of the viral RNA in 5 ul of test sample and the entire procedure is performed in one step.

### Components

The kit of this example consists of the following 4 tubes:

| | | Storage |
|---|---|---|
| | | Conditions |
| Tube No. | Component | (Short, Long term) |
| Tube 1 | RT-PCR Enzyme Mix | −20° C., −20° C. |
| | (e.g. from Qiagen) | |
| Tube 2 | RT-PCR Reaction Mix | 4° C., −20° C. |
| | (e.g. from Qiagen) | |
| Tube 3 | Primer Mix (Upper & Lower) | 4° C., −20° C. |
| | (30 μM each primer in 20 mM Tris, | |
| | 1 mM EDTA pH 8.2) | |
| Tube 4 | Positive Control | −20° C., −80° C. |
| | (RNA transcripts of the | |
| | gene targeted by the primers) | |

### Protocol, One Step RT-PCR

1. Sample Preparation

In a RNase-Free Eppendorf tube (0.5 ml or 0.2 ml size), add the following reagents per test/per reaction:

| Description | 50 μl/Rxn | 20 μl/Rxn |
|---|---|---|
| RT-PCR Enzyme Mix | 2.0 μl | 0.8 μl |
| RT-PCR Reaction Mix | 42.0 μl | 16.8 μl |
| Primer Mix (U & L) | 1.0 μl | 0.4 μl |
| RNA Samples | 5.0 μl | 2.0 μl |
| Total Volume | 50.0 μl | 20.0 μl |

2. Thermal Cycling Protocol-A

This thermal cycling protocol is used for three-block type PCR cyclers such as the RoboCycler® by Stratagene:

| | | | | |
|---|---|---|---|---|
| | Temp | | No. Of | |
| Stage | (° C.) | Duration | Cycle(s) | Step |

| | | | | | |
|---|---|---|---|---|---|
| 1 | 50 | 30 | mins | 1 | Reverse transcription |
| 2 | 95 | 15 | mins | 1 | Initial denaturation |
| 3 | 95 | 45 | secs | 42 | Denaturation |
| | 57 | 80 | secs | | Annealing |
| | 72 | 50 | secs | | Extention |
| 4 | 72 | 10 | mins | 1 | Final Extention |

3. Thermal Cycling Protocol-B

This thermal cycling condition is for one-block type PCR cycler such as the Px2 Thermal Cycler by Thermo Electron.

| | | | | |
|---|---|---|---|---|
| | Temp | | No. Of | |
| Stage | (° C.) | Duration | Cycle(s) | Step |

| | | | | | |
|---|---|---|---|---|---|
| 1 | 50 | 30 | mins | 1 | Reverse transcription |
| 2 | 95 | 15 | mins | 1 | Initial denaturation |
| 3 | 95 | 18 | secs | 42 | Denaturation |
| | 57 | 36 | secs | | Annealing |
| | 72 | 33 | secs | | Extention |
| 4 | 72 | 10 | mins | 1 | Final Extention |

It is preferred to put mineral oil in the wells of the thermocycler to maximize the conduction of the heat between a reaction tube and a well if it is necessary.
4. Termination of PCR Reaction

This step is optional.
(1) Add 30 μl of chloroform/tube. Vortex mix for 5 seconds.
(2) Centrifuge for 2 minutes. (Top=Aqueous phase, Bottom=Organic phase)
   The sample is reserved as the top, aqueous phase.
   5. Detection by Electrophoresis

The products of the PCR reactions are resolved by DNA gel electrophoresis by using 5 pi of the product reaction mixture product per lane. 3% agarose gel provides good resolution; gels are typically run at 100 V for 30 min.

The expected product size is 157bp when the IMCB-2 primer set of Example 1 is used. A result of such an assay is shown in FIG. 3C. This example shows the amplified product from the sample containing 5 copies/rxn (5 μl) SARS coronavirus RNA run in duplicate. The product was resolved by 3% agarose gel electrophoresis. 10% of the total reaction volume (5 μl) was loaded per lane. Lane 1, product of run 1; Lane 2, product of duplicate run; lane M, 100 bp ladder.

### EXAMPLE 5

### Specificity of RT-PCR Using the IMCB Primers

To verify that the primer sets designed in Example 1 can be used to detect SARS coronavirus specifically; the amplification of selected viruses is tested by RT-PCR using the IMCB primer sets 1 and 2. The following viruses are tested at the indicated titer to check the specificity of the IMCB RT-PCR primer sets 1 and 2:

| | | |
|---|---|---|
| Human Coronavirus 229E, | 2.8 × 10(6) | PFU/ml |
| ATCC VR-740 | | |
| Human Coronavirus OC43, | 3.5 × 10(7) | LD[50]/0.02 ml |
| ATCC VR-759 | | |
| Avian infectious bronchitis | 5.8 × 10(6) | PFU/ml |
| virus NCBI M95169 | | |
| Dengue virus, NCBI M87512 | 1.1 × 10(6) | PFU/ml |
| Yellow Fever virus, | 0.5 × 10(6) | PFU/ml |
| vaccine strain 17D | | |
| Human Enteric Coronavirus, | 0.2 × 10(6) | PFU/ml |
| ATCC VR-1475 | | |
| Bovine Coronavirus, ATCC VR-874 | 1.1 × 10(6) | PFU/ml |
| Rabbit Coronavirus, ATCC VR-920 | 23.3 × 10(6) | PFU/ml |
| Mouse hepatitis virus, | 0.6 × 10(6) | PFU/ml |
| ATCC VR-764 | | |
| Canine Coronavirus, ATCC VR-809 | 36.8 × 10(6) | PFU/ml |
| Rat Coronavirus, ATCC VR-1410 | 1.1 × 10(6) | PFU/ml |
| Feline-CoV RNA, ATCC VR-989 | 1 × 10(5.5) | CID[50]/ml |

The results in Table 1 show the IMCB primer pair specificity. IMCB primer pair 1 and IMCB primer pair 2 together with the ARTUS detection methods are used for detection of other Coronaviruses and viruses unrelated to SARS coronavirus. Negative results are observed for all the viruses using the IMCB-1 and IMCB-2 primer pairs. Thus, it is demonstrated that the IMCB pairs are highly specific to SARS coronavirus for detection.

TABLE 1
Specificity of IMCB-1 and IMCB-2 primer pairs

| Sample | Conc. | | 1st ARTUS | 2nd ARTUS | IMCB 1 | IMCB 2 |
|---|---|---|---|---|---|---|
| MRC5 RNA Ext | 2 | ug/ul | INCONCLUSIVE | Neg | Neg | Neg |
| MRC5 RNA Ext | 1 | ug/ul | Neg | na | Neg | Neg |
| Avian infectious bronchitis virus, NCBI M95169 | 5.8 × 10(6) | PFU/ml | Neg | na | Neg | Neg |
| Dengue virus, NCBI M87512 | 1.1 × 10(6) | PFU/ml | Neg | na | Neg | Neg |
| Yellow Fever virus, vaccine strain 17D | 0.5 × 10(6) | PFU/ml | Neg | na | Neg | Neg |
| Human Enteric coronavirus, ATCC VR-1475 | 0.2 × 10(6) | PFU/ml | Neg | na | Neg | Neg |
| Bovine coronavirus, ATCC VR-874 | 1.1 × 10(6) | PFU/ml | Neg | na | Neg | Neg |
| Rabbit coronavirus, ATCC VR-920 | 23.3 × 10(6) | PFU/ml | Neg | na | Neg | Neg |
| Mouse hepatitis virus, ATCC VR-764 | 0.6 × 10(6) | PFU/ml | Neg | na | Neg | Neg |
| Canine coronavirus, ATCC VR-809 | 36.8 × 10(6) | PFU/ml | Neg | na | Neg | Neg |
| Rat coronavirus, ATCC VR-1410 | 1.1 × 10(6) | PFU/ml | Neg | na | Neg | Neg |
| Human coronavirus 229E, ATCC VR-740 | 2.8 × 10(6) | PFU/ml | Neg | na | Neg | Neg |

### EXAMPLE 6

### Comparison of the Sensitivity of the Available SARS Coronavirus Detection Kits

The available SARS detection kits available, Eiken, Artus and Roche SARS diagnostics, are compared with the three IMCB primer pairs in their ability to detect the same virus standard. The Eiken kit is tested using a one step RT-Lamp. (T. Notomi et al., “Loop-mediated Isothermal Amplification of DNA”, Nucleic Acids Research 15:E63 (2000).) The IMCB-1 primer pairs are tested using the concentrations of reagents set forth in Example 2. However the reagent materials are those described in Example 3 and the PCR protocol was that of the one step RT-PCR described in Example 3. The IMCB-2 primer pairs are tested using the reagent materials and concentrations and the one step RT-PCR protocol described in Example 3. The amplification products that are obtained using the IMCB-1 and IMCB-2 primer sets are analysed by agarose gel electrophoresis with ethidium bromide staining. The IMCB-3 primer pair is tested using a one step RT-PCR kit prototype optimized for the ABI 7000 Real Time system using a Taqman™ probe described in Example 1. The Artus ABI kit is tested using RealArt™ HPA-Coronavirus TM RT PCR, Abbot List No. B3K360 REV.2003-10-R2. (10 μl sample is used per reaction instead of 5 μl). The Artus light cycler kit is tested using RealArt™ HPA-Coronavirus LC RT PCR Reagent (Cat No: 5601-03). The Roche kit is tested using Light Cycler™ SARS Quantification kit (cat 03604438001) Version 1.

Table 2 shows the results of the comparative testing. The viral sample copy numbers used is decreased from 83 copies per 5 μl at the top of the table to 0.1 copies per 5 μl at the bottom of the table in column 1 under the sample volume/run (vol/rxn). Across the table the numbers in brackets behind Pos indicate how many positive detections there are per the total number of tests done.

From Table 2 it is seen that the IMCB-2 primer sets provide the most sensitive detection; the IMCB-1 and -2 primer pairs can reliably detect as little as 0.8 copies of viral nucleic acid per 5 μl sample.

TABLE 2
Detection of SARS-CoV by BKEN, IMCB, ARTUS, and ROCHE Systoms

| | BKEN LAMP | | IMCB 1 | IMCB 2 | IMCB 3 (ABI) | |
|---|---|---|---|---|---|---|
| Sample code | Run 1 | Run 2 | Run 1 | Run 1 | Run 1 | Run 2 |
| Sample vol/rxn | 5 ul | 5 ul | 5 ul | 5 ul | 5 ul | 5 ul |
| (N-5.6) | Pos(8/8) | Pos(6/6) | Pos (12/12) | Pos (8/8) | | |
| 83 copies/5 ul | | | | | | |
| (N-6.0) | Pos(8/8) | Pos(6/6) | Pos (12/12) | Pos (8/8) | Pos (12/12) | Pos (6/6) |
| 26 copies/5 ul | | | | | | |
| (N-6.5) | (not done) | Pos(4/6) | Pos (12/12) | Pos (8/8) | Pos (12/12) | Pos (6/6) |
| 8 copies/5 ul | | | | | | |
| (N-7.0) | Pos(4/8) | Pos(4/6) | Pos (12/12) | Pos (8/8) | Pos (12/12) | Pos (8/6) |
| 3 copies/5 ul | | | | | | |
| (N-7.5) | | Pos(0/6) | Pos (12/12) | Pos (8/8) | Pos (10/12) | Pos (6/6) |
| 0.8 copies/5 ul | | | | | | |
| (N-8.0) | Pos(0/8) | | Pos (4/10) | Pos (1/8) | Pos (2/12) | Pos (1/6) |
| 0.3 copies/5 ul | | | | | | |
| (N-8.5) | | | w Pos (2/10) | P(2/6) | Pos (1/12) | Pos (1/6) |
| 0.1 copies/5 ul | | | | | | |

| | | | [symbol] | | | |
|---|---|---|---|---|---|---|
| | [symbol] | [symbol] | Run 2 | | | ROCHE |
| Sample code | Run 1 | Run 1 | 5 ul | [symbol] | [symbol] | Run 1 |
| Sample vol/rxn | 10 ul | 5 ul | Average (copy/ul) | | STDV, CV | 5 ul |
| (N-5.6) | | 13.080 | 13.460 | 12.164 | 0.923 | |
| 83 copies/5 ul | | 12.970 | 11.740 | Pos (7/7 | 7.6% | |
| | | 10.840 | 11.360 | | | |
| | | 11.700 | | | | |
| (N-6.0) | | 5.236 | 6.114 | 5.215 | 0.457 | Pos(8/8) |
| 26 copies/5 ul | | 5.327 | 4.534 | Pos(7/7) | 8.8% | |
| | | 5.192 | 4.808 | | | |
| | | 5.296 | | | | |
| (N-6.6) | | 2.894 | 2.094 | 2.520 | 0.479 | (not done) |
| 8 copies/5 ul | | 3.335 | 2.780 | Pos (7/7 | 19.0% | |
| | | 2.491 | 2.202 | | | |
| | | 1.847 | | | | |
| (N-7.0) | Pos (2/2) | 0.354 | 0.472 | 0.393 | 0.232 | Pos(0/8) |
| 3 copies/5 ul | | 0.672 | 0.192 | Pos (7/7 | 59.0% | |
| | | 0.700 | 0.549 | | | |
| | | 0.013 | 0.191 | | | |
| (N-7.5) | Pos (2/2) | 0.417 | 0.219 | 0.413 | 0.318 | |
| 0.8 copies/5 ul | | 0.000 | 0.982 | Pos(6/7) | 77.0% | |
| | | 0.768 | 0.192 | | | |
| | | 0.324 | | | | |
| (N-8.0) | Pos (0/2) | 0.000 | 0.000 | 0.014 | 0.034 | |
| 0.3 copies/5 ul | noise* | 0.000 | 0.097 | Pos(1/7) | 244.9% | |
| | | 0.000 | 0.000 | | | |
| | | 0.000 | | | | |
| (N-8.5) | | 0.000 | 0.000 | 0.061 | 0.137 | |
| 0.1 copies/5 ul | | 0.368 | 0.000 | Pos(1/6) | 223.6% | |
| | | 0.000 | 0.000 | | | |

*background noise moderately high
BKEN: One step RT-LAMP
IMCB 1: One step RT-PCR with Primer set #1, product was analysed by Agarose gel/BBr.
IMCB 2: One step RT-PCR with Primer set #2, product was analysed by Agarose gel/BBr.
IMCB 3: (ABI): One step RT-PCR Kit prototype optimized for ABI 7000 Real Time System using TaqVan Probe
ARTUS (ABI): Real HPA-Coronavirus TM RT PCR, ABBOT List. No. B3K360 REV.2003-10-R2.
Note:
10 ul sample was used per reaction instead of 5 ul.
ARTUS: RealArt HPA-Coronavirus LC RT PCR Reagens (cat No: 5601-03)
ROCHE Light Cycler SARS Quantification Kit (cat 03604438001) Version 1

### EXAMPLE 7

### Analysis of Patient Samples

Clinical samples are obtained from a number of patients and are analyzed by the assay of the invention, using primer set IMCB-2 and the assay method described in Example 3. The assay method described in Example 4 is used for samples analysed by Artus using their PCR kit. Results are shown in Table 3.

Column 1 in Table 3 is a sample identification number. Column 2 is the description of the type of sample that was taken from the patients. Columns 3 and 4 indicate the results of the Artus detection method. Column 5 is the results of the detection using RT PCR with IMCB-2 primer set. The final column 6 documents notes referring to each sample.

TABLE 3
Analysis of patient samples

| | IMCB | | 1ST | 2ND | PS 2 | |
|---|---|---|---|---|---|---|
| | ID | DESC | ARTUS | ARTUS | IMCB | IMCB Note |
| | P65 | PLASMA | N | | N | |
| | P66 | STOOL | N | | N | |
| | P67 | STOOL | 247 | | N | |
| | P68 | STOOL | N | | N | |
| | P69 | STOOL | 194 | | N | |
| | P70 | STOOL | N | | N | |
| | P71 | STOOL | N | | POS | a−>g (4656) |
| | P72 | STOOL | N | | N | |
| | P73 | STOOL | N | | N | |
| | P74 | STOOL | N | | N | |
| | P75 | STOOL | N | | N | |
| | P76 | THROAT SWAB | N | | N | |
| | P77 | STOOL | N | | N | |
| | P78 | BAL | N | | N | |
| | P79 | SALIVA | N | | N | |
| ) | P80 | SERUM | N | | N | |
| | P81 | PLASMA | N | | N | |
| | P82 | STOOL | INC | | N | |
| | P83 | SPUTUM | N | | N | |
| | P84 | SPUTUM | N | | N | |
| | P85 | STOOL | N | | N | |
| | P86 | SALIVA | N | | N | |
| | P87 | PLASMA | N | | N | |
| | P88 | STOOL | N | | N | |
| | P89 | STOOL | N | | N | |
| | P90 | STOOL | N | | N | |
| | P91 | SPUTUM | N | | N | |
| | P92 | SPUTUM | N | | N | |
| | Q1 | SERUM | N | | N | |
| | Q2 | STOOL | N | | N | |
| | Q3 | STOOL | N | | N | |
| ) | Q4 | STOOL | INC | | N | |
| | Q5 | STOOL | N | | N | |
| | Q6 | STOOL | N | | N | |
| | Q7 | STOOL | N | | N | |
| | Q8 | STOOL | N | | N | |
| | Q9 | SERUM | N | | N | |
| | Q10 | PLASMA | N | | N | |
| | Q11 | STOOL | N | | N | |
| | Q12 | STOOL | 575 | 768 | N | |
| | Q13 | STOOL | 68 | | N | |
| | Q14 | STOOL | INC | | N | |
| | Q15 | STOOL | N | | N | |
| | Q16 | STOOL | N | | N | |
| | Q17 | STOOL | N | | N | |
| | Q18 | STOOL | 206 | 1,423 | POS | a−>g (4656) |
| | Q19 | PLASMA | N | | N | |
| | Q20 | SERUM | N | | N | |
| | Q21 | PLASMA | N | | N | |
| | Q22 | STOOL | N | N | POS | a−>g (4656) |
| | Q23 | STOOL | 456 | 325 | POS | a−>g (4656) |
| | Q24 | STOOL | 453 | 477 | N | |
| | Q25 | STOOL | 236 | N | N | |
| | Q26 | STOOL | 1,083 | 719 | N | |
| | Q27 | STOOL | 786 | 469 | N | |
| | Q28 | Stool: 2 Extract | 850 | | N | |
| | Q29 | Stool: 2 Extract | N | | POS | a−>g (4656) |
| | Q30 | Stool: 2 Extract | N | | N | |
| | Q31 | Stool: 2 Extract | INC | | N | |
| | Q32 | Stool: 2 Extract | N | | POS | a−>g (4656) |
| | Q33 | Stool: 2 Extract | N | | N | |
| | Q34 | Stool | 1,926 | 519 | N | |
| | Q35 | Stool | 153 | 277 | POS | a−>g (4656) |
| | Q36 | Serum | N | | N | |
| | Q37 | Stool: 2 Extract | 1,253 | | N | |
| | Q38 | Stool: 2 Extract | N | | N | |
| | Q39 | QS-1 | ? | | N | |
| | Q40 | RNA EXRACT + ct | ? | | N | LowerBand (not SA [symbol] |
| | Q41 | 2nd extract | 606 | | POS | a−>g (4656) |
| | Q42 | 2nd extract | N | | N | |
| | Q43 | stool | 193 | N | POS | a−>g (4S56) |
| | Q44 | stool | 776 | 887 | N | |
| | Q45 | Old PBS | 250 | | N; N, N | repeat ×2 negative |
| | Q46 | New PBS | N | | N, N, N | repeat ×2 negative |
| | Q47 | EHI + RNA Ex Cor | 500 | | POS | Urbani, NEA |
| | Q48 | PLASMA | N | | N | |
| | Q49 | STOOL | N | | N | |
| | Q50 | PLASMA | N | | N | |
| | Q51 | SURERNATANT | N | | N | |
| | Q52 | CELL PELLET | N | | N | |
| | Q53 | STOOL | N | | N | |
| | Q54 | STOOL | N | | N | |
| | Q55 | STOOL | N | | POS | a−>g (4656) |
| | Q56 | SERUM | N | | N | |
| | Q57 | SPUTUM (SUP) | N | | N | |
| | Q58 | SPUTUM (C.P) | N | | POS | a−>g (4656) |
| | Q59 | STOOL | N | | N | |
| | Q60 | PLASMA | N | | N | |
| | Q61 | STOOL | N | | N | |
| | Q62 | SPUTUM (SUP) | N | | POS | a−>g (4656) |
| | Q63 | SPUTUM (C.P) | N | | N | |
| | Q64 | STOOL | N | | N | |
| | Q65 | STOOL | N | | POS | a−>g (4656) |
| | Q66 | STOOL | N | | N | |
| | Q67 | STOOL | N | | N | |
| | Q68 | PLASMA | N | | N | |
| | Q69 | STOOL | N | | N | |
| | Q70 | SPUTUM | N | | POS | (see Q58) |
| | Q71 | SPUTUM | N | | N | |
| | Q72 | STOOL | N | | N | |
| | Q73 | SPUTUM | N | | N | |
| | Q74 | SPUTUM | INC | | N | |
| | Q75 | STOOL | N | | N | |
| | Q76 | INHIBITOR TEST | (POS) | | POSPOSPOS | Urbani, NEA |
| | Q77 | INHIBITOR TEST | (POS) | | POSPOSPOS | Urbani, NEA |

### EXAMPLE 8

### SARS Real-Time PCR Diagnostic Kit (RT-PCR)

A kit of this example is typically prepared to contain 50 or 100 reactions. This Real-Time Reverse Transcriptase-Polymerase Chain Reaction (RT-PCR) kit is optimized to detect the presence of Severe Acute Respiratory syndrome Coronavirus Ribonucleic Acid (SARS coronavirus RNA)in a biological sample. This kit is optimized for use with the Applied Biosystems Real-Time PCR, ABI Prism 7500, but may be used with other suitable detection platforms as described elsewhere herein.

The portion of the SARS coronavirus genome amplified by the kit lies in the proteinase region of the SARS coronavirus RNA. The kit is sufficiently sensitive to detect a few molecules of RNA in each RT-PCR reaction.

### Components

This kit of this Example consists of the following 4 tubes:

Tube 1 : Reaction Mix (e.g. ABI cat. No. 4309169)
Tube 2 : Enzyme Mix (e.g. ABI cat. No. 4309169)
Tube 3 : Probe Mix (3 μM upper primer, 3 μM lower primer, 2 μM probe in 20 mM Tris, 1 mM EDTA pH 8.2)
Tube 4 : Positive Control (RNA transcripts of the gene targeted by the primers)

### Protocol

1. RT-PCR

The following reaction mix is prepared in a 96-well Optical plate:

| Tube No. | Description | Vol/Rxn |
|---|---|---|
| Tube 1 | Reaction Mix | 25.0 μl |
| Tube 2 | Enzyme Mix | 1.25 μl |
| Tube 3 | Probe Mix | 5.0 μl |
| Tube 4 | Distilled Water | 13.75 μl |
| — | RNA Sample | 5.0 μl |
| | Total Volume | 50.0 μl |

Caution should be taken to avoid contamination.

2. —Thermal Cycling Conditions:

| | | | |
|---|---|---|---|
| | Temp | | No. of |
| Step | (° C.) | Duration | Cycle(s) |

| | | | | |
|---|---|---|---|---|
| 1 | 48 | 30 | mins | 1 |
| 2 | 95 | 10 | mins | 1 |
| 3 | 95 | 15 | secs | 50 |
| 4 | 60 | 60 | secs | 1 |

The IMCB-3 primer set and probe are tested for their ability to detect SARS coronavirus using the Stratagene real-time PCR system Mx3000P. The system is used according to the manufacturer's instructions on samples from infected patients. The samples are diluted several fold to a total of viral copy number per 5 μl ranging between 7.5 to 6.

Results are shown in Table 4. The number of viral copies of SARS coronavirus per 5 μl ranges from 7.5 to 6 in all three runs. Row A (A1-3) is a control no virus sample. Row C (C1-C10) is detection of samples at 7.5 viral copies 5 μl, Row E (E1-E10) is detection of samples at 7.5 viral copies 5 μl, and Row G (G1-G10) is detection of samples at 7.5 viral copies 5 μl. The 4^{th} column indicates how many positive results are detected per number of samples tested. The results demonstrate that the IMCB-3 primer set and probe provide a sensitive and specific assay for SARS coronavirus that is useful in a clinical setting.

TABLE 4
Sensitivity of the IMCB-3 primer set in real-time PCR analysis

| | | | | |
|---|---|---|---|---|
| Well | Well Name | Well Type | Threshold (dR) | Ct (dR) |

Run #1:
r

| | | | | | |
|---|---|---|---|---|---|
| A1 | — | NTC | 2907.809 | No Ct | 0/3 |
| A2 | — | NTC | 2907.809 | No Ct | |
| A3 | — | NTC | 2907.809 | No Ct | |
| C1 | BMRC | −7.5 | Unknown 2907.809 | 41.67 | 8/10 |
| C2 | BMRC | −7.5 | Unknown 2907.809 | No Ct | |
| C3 | BMRC | −7.5 | Unknown 2907.809 | No Ct | |
| C4 | BMRC | −7.5 | Unknown 2907.809 | 40.9 | |
| C5 | BMRC | −7.5 | Unknown 2907.809 | 39.81 | |
| C6 | BMRC | −7.5 | Unknown 2907.809 | 40.09 | |
| C7 | BMRC | −7.5 | Unknown 2907.809 | 41.16 | |
| C8 | BMRC | −7.5 | Unknown 2907.809 | 42.54 | |
| C9 | BMRC | −7.5 | Unknown 2907.809 | 40.37 | |
| C10 | BMRC | −7.5 | Unknown 2907.809 | 40.19 | |
| E1 | BMRC | −7.0 | Unknown 2907.809 | 39.37 | 10/10 |
| E2 | BMRC | −7.0 | Unknown 2907.809 | 40.1 | |
| E3 | BMRC | −7.0 | Unknown 2907.809 | 41.86 | |
| E4 | BMRC | −7.0 | Unknown 2907.809 | 40.77 | |
| E5 | BMRC | −7.0 | Unknown 2907.809 | 40.13 | |
| E6 | BMRC | −7.0 | Unknown 2907.809 | 39.29 | |
| E7 | BMRC | −7.0 | Unknown 2907.809 | 40.26 | |
| E8 | BMRC | −7.0 | Unknown 2907.809 | 39.59 | |
| E9 | BMRC | −7.0 | Unknown 2907.809 | 42.04 | |
| E10 | BMRC | −7.0 | Unknown 2907.809 | 41.12 | |
| G1 | BMRC | −6.5 | Unknown 2907.809 | 37.02 | 10/10 |
| G2 | BMRC | −6.5 | Unknown 2907.809 | 37.64 | |
| G3 | BMRC | −6.5 | Unknown 2907.809 | 39.05 | |
| G4 | BMRC | −6.5 | Unknown 2907.809 | 38.24 | |
| G5 | BMRC | −6.5 | Unknown 2907.809 | 37.39 | |
| G6 | BMRC | −6.5 | Unknown 2907.809 | 37.38 | |
| G7 | BMRC | −6.5 | Unknown 2307.809 | 38.26 | |
| G8 | BMRC | −6.5 | Unknown 2907.809 | 37.75 | |
| G9 | BMRC | −6.5 | Unknown 2907.809 | 37.37 | |
| G10 | BMRC | −6.5 | Unknown 2907.809 | 38.45 | |

Run #2
report 040902

| | | | | | |
|---|---|---|---|---|---|
| A1 | — | NTC | 2929.155 | No Ct | 0/3 |
| A2 | — | NTC | 2929.155 | No Ct | |
| A3 | — | NTC | 2929.155 | No Ct | |
| C1 | BMRC | −7.5 | Unknown 2929.155 | 41.05 | 9/10 |
| C2 | BMRC | −7.5 | Unknown 2929.155 | 41 | |
| C3 | BMRC | −7.5 | Unknown 2929.155 | 42.06 | |
| C4 | BMRC | −7.5 | Unknown 2929.155 | 42.32 | |
| C5 | BMRC | −7.5 | Unknown 2929.155 | 42.22 | |
| C6 | BMRC | −7.5 | Unknown 2929.155 | 41.92 | |
| C7 | BMRC | −7.5 | Unknown 2929.155 | 41.45 | |
| C8 | BMRC | −7.5 | Unknown 2929.155 | 40.72 | |
| C9 | BMRC | −7.5 | Unknown 2929.155 | No Ct | |
| C10 | BMRC | −7.5 | Unknown 2929.155 | 42.39 | |
| E1 | BMRC | −7.0 | Unknown 2929.155 | 42.03 | 9/10 |
| E2 | BMRC | −7.0 | Unknown 2929.155 | 41.15 | |
| E3 | BMRC | −7.0 | Unknown 2929.155 | 41.11 | |
| E4 | BMRC | −7.0 | Unknown 2929.155 | 19.2 | |
| E5 | BMRC | −7.0 | Unknown 2929.155 | No Ct | |
| E6 | BMRC | −7.0 | Unknown 2929.155 | 39.58 | |
| E7 | BMRC | −7.0 | Unknown 2929.155 | 43.15 | |
| E8 | BMRC | −7.0 | Unknown 2929.155 | 39.63 | |
| E9 | BMRC | −7.0 | Unknown 2929.155 | 40.38 | |
| E10 | BMRC | −7.0 | Unknown 2929.155 | 40.86 | |
| G1 | BMRC | −6.5 | Unknown 2929.155 | 38.57 | 10/10 |
| G2 | BMRC | −6.5 | Unknown 2929.155 | 39.11 | |
| G3 | BMRC | −6.5 | Unknown 2929.155 | 38.63 | |
| G4 | BMRC | −6.5 | Unknown 2929.155 | 38.14 | |
| G5 | BMRC | −6.5 | Unknown 2929.155 | 37.77 | |
| G6 | BMRC | −6.5 | Unknown 2929.155 | 38.33 | |
| G7 | BMRC | −6.5 | Unknown 2929.155 | 39.1 | |
| G8 | BMRC | −6.5 | Unknown 2929.155 | 38.08 | |
| G9 | BMRC | −6.5 | Unknown 2929.155 | 39.07 | |
| G10 | BMRC | −6.5 | Unknown 2929.155 | 39.04 | |

Run #3
report 040903

| | | | | | |
|---|---|---|---|---|---|
| A1 | — | NTC | 3062.797 | No Ct | 0/3 |
| A2 | — | NTC | 3962.797 | No Ct | |
| A3 | — | NTC | 3962.797 | No Ct | |
| C1 | BMRC | −7.5 | Unknown 3962.797 | 43.21 | 5/10 |
| C2 | BMRC | −7.5 | Unknown 3962.797 | 40.46 | |
| C3 | BMRC | −7.5 | Unknown 3962.797 | 42.1 | |
| C4 | BMRC | −7.5 | Unknown 3962.797 | No Ct | |
| C5 | BMRC | −7.5 | Unknown 3962.797 | 41.78 | |
| C6 | BMRC | −7.5 | Unknown 3962.797 | 40.7 | |
| C7 | BMRC | −7.5 | Unknown 3962.797 | No Ct | |
| C8 | BMRC | −7.5 | Unknown 3962.797 | No Ct | |
| C9 | BMRC | −7.5 | Unknown 3962.797 | No Ct | |
| C10 | BMRC | −7.0 | Unknown 3962.797 | No Ct | |
| E1 | BMRC | −7.0 | Unknown 3962.797 | 38.4 | 10/10 |
| E2 | BMRC | −7.0 | Unknown 3962.797 | 40.14 | |
| E3 | BMRC | −7.0 | Unknown 3962.797 | 40.03 | |
| E4 | BMRC | −7.0 | Unknown 3962.797 | 39.04 | |
| E5 | BMRC | −7.0 | Unknown 3962,797 | 41.12 | |
| E6 | BMRC | −7.0 | Unknown 3962.797 | 39.48 | |
| E7 | BMRC | −7.0 | Unknown 3962.797 | 39.55 | |
| E8 | BMRC | −7.0 | Unknown 3962.797 | 39.89 | |
| E9 | BMRC | −7.0 | Unknown 3962.797 | 39.57 | |
| E10 | BKRC | −7.0 | Unknown 3962.797 | 42.92 | |
| G1 | BMRC | −6.5 | Unknown 3962.797 | 38.41 | 10/10 |
| G2 | BMRC | −6.5 | Unknown 3962.797 | 37.41 | |
| G3 | BMRC | −6.5 | Unknown 3962.797 | 38.12 | |
| G4 | BMRC | −6.5 | Unknown 3962.797 | 37.73 | |
| G5 | BMRC | −6.5 | Unknown 3962.797 | 37.6 | |
| G6 | BMRC | −6.5 | Unknown 3962.797 | 38.58 | |
| G7 | BMRC | −6.5 | Unknown 3962.797 | 37.9 | |
| G8 | BMRC | −6.5 | Unknown 3962.797 | 38.38 | |
| G9 | BMRC | −6.5 | Unknown 3962.797 | 38.27 | |
| G10 | BMRC | −6.5 | Unknown 3962.797 | 38.1 | |

It will be understood by those of skill in the art that the presence or absence of components in the above method, the concentrations of chemicals, the cycling conditions, and the equipment can be modified to suit the particular needs and to optimize reaction conditions.

Moreover, those of skill in the art will recognize that the above method and the above description can encompass modifications that fall within the spirit and scope of the instant invention.

### REFERENCES

The following references are cited in the instant specification. Each of the following references is hereby incorporated by reference in its entirety and for all purposes by such citation:
Drosten, C., et al., 2003. Identification of a novel coronavirus in patients with severe acute respiratory syndrome. N. Engl. J. Med. 348:1967-1976.
Notomi, T. et al., 2000. Loop-mediated isothermal amplification of DNA. Nucleic Acids Research 15:E63.
Rota, P. A., et al. 2003. Characterization of a novel coronavirus associated with severe acute respiratory syndrome. Science 300:1394-1399.
Ruan, Y. J., et al., 2003. Comparative full-length genome sequence analysis of 14 SARS coronavirus isolates and common mutations associated with putative origins of infection. Lancet. 361, 1779-1785.
WHO Update 71. Status of diagnostic tests, training course in China. http://www.who.int/entity/csr/don/2003_{—}06_{—}02 a/en

1. A method for detecting SARS coronavirus nucleic acid in a sample comprising:
A) amplifying a nucleic acid of said sample with a reverse transcriptase and at least one primer specific for a NSP1 region of a SARS coronavirus to generate a nucleic acid ampification product; and
B) analyzing said amplification product wherein detecting an expected nucleic acid amplification product indicates the presence of SARS coronavirus nucleic acid in the sample.

2. The method of claim 1, wherein the at least one primer specific for the NSP1 region of the SARS coronavirus is a primer consisting essentially of a sequence selected from the group consisting of SEQ ID NOs: 3, 4, 6, 7, 9 and 10.

3. The method of claim 1, wherein the at least one primer specific for the NSP1 region of the SARS coronavirus is a primer consisting of a sequence selected from the group consisting of SEQ ID NOs: 3, 4, 6, 7, 9 and 10.

4. The method of claim 1, wherein a primer having the nucleotide sequence of SEQ ID NO: 3 and a primer having the nucleotide sequence of SEQ ID NO: 4 are used and the expected nucleic acid amplification product is detected as a polynucleotide that is 352 nucleotides long.

5. The method of claim 1, wherein a primer having the nucleotide sequence of SEQ ID NO: 6 and a primer having the nucleotide sequence of SEQ ID NO: 7 are used and the expected nucleic acid amplification product is detected as a polynucleotide that is 157 nucleotides long.

6. The method of claim 1, wherein a primer having the nucleotide sequence of SEQ ID NO: 9 and a primer having the nucleotide sequence of SEQ ID NO: 10 are used and the expected nucleic acid amplification product is detected as a polynucleotide that is 77 nucleotides long.

7. The method of any one of claims 1 to 6, wherein said nucleic acid amplification product is analyzed by chromatography.

8. The method of claim 7 wherein the nucleic acid amplification is performed by a polymerase chain reaction.

9. A method for detecting SARS coronavirus nucleic acid in a sample comprising:
A) amplifying a nucleic acid of said sample with a reverse transcriptase and a first primer having the sequence of SEQ ID NO: 9 and a second primer having the sequence of SEQ ID NO: 10 to generate a nucleic acid amplification product;
B) detecting said nucleic acid amplification product by specific hybridization of a probe having the sequence of SEQ ID NO: 11;
wherein detection of a specifically hybridizing amplified nucleic acid fragment indicates the presence of SARS coronavirus nucleic acid in the sample.

10. The method of claim 9, in which the nucleic acid amplification is performed by a polymerase chain reaction.

11. A method for detecting the presence of SARS coronavirus nucleic acid in a sample, comprising:
i) amplifying nucleic acids present in the sample using a forward primer and a reverse primer selective for the region of the SARS coronavirus genome from nucleotide 6652 to nucleotide 7003, said primers having a certain primer length in nucleotides and being separated by a separation length that is a certain number of nucleotides, to obtain an amplification product;
ii) determining the length of the amplification product in nucleotides;
iii) the presence of an amplification product having a length in nucleotides that is the sum of the forward primer length, the reverse primer length and the separation length indicating the presence of SARS coronavirus nucleic acid in the sample.

12. The method of claim 12, in which the nucleic acid amplification is performed by a polymerase chain reaction.

13. A method for detecting the presence of SARS coronavirus nucleic acid in a sample, comprising:
i) amplifying nucleic acids present in the sample using a forward primer and a reverse primer selective for the region of the SARS coronavirus genome from nucleotide 6652 to nucleotide 7003 to obtain an amplification product; and
ii) detecting the amplification product by specific hybridization of a probe having a nucleotide sequence of at least 18 contiguous nucleotides of the portion of the SARS coronavirus genome from nucleotide 6652 to 7003;
specific hybridization of the probe to the amplification product indicating the presence of SARS nucleic acid in the sample.

14. The method of claim 13, in which the nucleic acid amplification is performed by a polymerase chain reaction.

15. A method for detecting the presence of SARS coronavirus nucleic acid in a sample, comprising:
i) amplifying nucleic acids present in the sample using a forward primer and a reverse primer selective for the region of the SARS coronavirus genome from nucleotide 4609 to nucleotide 4765, said primers having a certain primer length in nucleotides and being separated by separation length that is a certain number of nucleotides, to obtain an amplification product;
ii) determining the length of the amplification product in nucleotides;
iii) the presence of an amplification product having a length in nucleotides that is the sum of the forward primer length, the reverse primer length and the separation length indicating the presence of SARS coronavirus nucleic acid in the sample.

16. The method of claim 15, in which the nucleic acid amplification is performed by a polymerase chain reaction.

17. A method for detecting the presence of SARS coronavirus nucleic acid in a sample, comprising:
i) amplifying nucleic acids present in the sample using a forward primer and a reverse primer selective for the region of the SARS coronavirus genome from nucleotide 4609 to nucleotide 4765 to obtain an amplification product; and
ii) detecting the amplification product by specific hybridization of a probe having a nucleotide sequence of at least 18 contiguous nucleotides of the portion of the SARS coronavirus genome from nucleotide 4609 to 4765;
specific hybridization of the probe to the amplification product indicating the presence of SARS coronavirus nucleic acid in the sample.

18. The method of claim 17, in which the nucleic acid amplification is performed by a polymerase chain reaction.

19. A SARS coronavirus detection kit comprising at least one primer for a nucleic acid amplification reaction selected from the group consisting of SEQ ID NOs: 3, 4, 6, 7, 9 and 10.

20. The SARS coronavirus detection kit according to claim 19, that comprises at least one pair of primers selected from the group consisting of a primer having the sequence of SEQ ID NO: 3 and a primer having the sequence of SEQ ID NO: 4, a primer having the sequence of SEQ ID NO: 6 and a primer having the sequence of SEQ ID NO: 7, and a primer having the sequence of SEQ ID NO: 9 and a primer having the sequence of SEQ ID NO: 10.

21. The SARS coronavirus detection kit according to claim 19 or 20, that also comprises a SARS coronavirus genomic nucleic acid, or at least a portion thereof comprising the NSP1 region.

22. The SARS coronavirus detection kit according to claim 21, in which the SARS coronavirus genomic nucleic acid has the nucleotide sequence of SEQ ID NO: 1, or the RNA equivalent thereof.

23. Use of an oligonucleotide having a sequence selected from the group consisting of SEQ ID NOs: 3, 4, 6, 7, 9, 10 and 11, in a method for detecting the presence of SARS coronavirus nucleic acids in a sample.
